(19) United States
(12) Patent Application Publication
(10) Pub. No.: US 2014/0236114 A1
(43) Pub. Date: Aug. 21, 2014

(54) Disposable Absorbent Article Having Serviceable Indicia Indicating Improper Fit

(71) Applicant: THE PROCTER & GAMBLE COMPANY, CINCINNATI, OH (US)

(72) Inventors: Lisa Jane GOODLANDER, Okeana, OH (US); Donald Carroll ROE, West Chester, OH (US)

(73) Assignee: THE PROCTER & GAMBLE COMPANY, CINCINNATI, OH (US)

(21) Appl. No.: 14/264,171

(22) Filed: Apr. 29, 2014

Related U.S. Application Data
(63) Continuation of application No. 11/303,306, filed on Dec. 16, 2005.

(51) Int. Cl. A61F 13/49; A61F 13/56

(52) U.S. Cl. CPC A61F 13/4902; A61F 13/5638; A61F 13/5655; A61F 2013/49041
USPC 604/385.29

(57) ABSTRACT

A disposable absorbent article worn about the lower torso of a wearer includes at least one serviceable indicium that facilitates the process of fitting the absorbent article to the wearer by providing an apparent visual and physical indication when the article is not properly fitted. The apparent visual and physical indication includes a detectable distortion of the waist opening, the leg openings or both the waist opening and the leg openings. The detectable distortion includes at least one serviceable indicium disposed on a portion of the waist opening or leg opening or both that becomes flipped in towards an inner surface of the article or flipped out towards the outer surface of the article when the article is not properly fitted.

### CROSS REFERENCE TO RELATED APPLICATION

This application is a continuation of, and claims priority under 35 U.S.C. §120 to, U.S. patent application Ser. No. 11/303,306, filed on Dec. 16, 2005, the entire disclosure of which is hereby incorporated by reference.

### FIELD OF THE INVENTION

This invention is directed to hygienic absorbent articles, such as diapers, training pants and the like. Particularly, the invention is directed to disposable diapers designed to facilitate the process of fitting the diaper to a wearer.

### BACKGROUND OF THE INVENTION

Absorbent articles are well known in the art. These articles typically have an absorbent core held or positioned in proximity to the body of a wearer during use by a fastening system in order to capture and absorb bodily exudates discharged from the wearer. Typical absorbent articles include a topsheet facing the wearer, which permits fluid exudates to pass through, and a backsheet, which prevents the exudates from escaping from the absorbent article.

Disposable absorbent articles such as diapers are designed to absorb and contain bodily waste in order to prevent soiling of the body and clothing of the wearer. The disposable diapers typically comprise a single design available in different sizes to fit a variety of wearers ranging from newborns to toddlers undergoing toilet training. The design of the diaper typically affects performance, such as, ability to absorb and contain bodily waste. The size of the diaper typically affects fit, for example, the size of the diaper waist opening, the size of the openings around the thighs, and the length or “pitch” of the diaper.

Articles worn externally to the body of the wearer, such as diapers, are commonly misapplied due to awkward positioning of the wearer or the restless movement of the wearer during fitting. Such misapplication may result in an uneven fit; gaps, which result in leakage; and misplaced parts (such as fasteners), which may result in marking the skin of the wearer and/or discomfort. This is particularly true of caregivers dealing with uncooperative wearers such as babies. Babies, even from a young age, move their legs into awkward positions, roll from side to side, or even violently resist diaper changes using hand and leg motions. As a result, the caregiver often has to hold portions of the wearer's body as well as the diaper during the change process making it very difficult to achieve proper alignment of the diaper for fitting.

Attempts have been made in the art to improve the process of applying an absorbent article to the wearer such as by using serviceable indicia such as described in co-pending U.S. Patent Application Publication 2003/0158532A1. The serviceable indicia described in the publication are features consisting of various patterns that assist in the application of articles worn primarily externally on the body of the wearer, especially hygienic absorbent articles, such as diapers, adult incontinence articles, feminine protection articles and the like. While the serviceable indicia provide a visual aid which facilitates fitting the article on the wearer via alignment, they do not necessarily ensure that all of the features are properly oriented for contact with the wearer once the fitting is complete. For instance, a leg or waist opening may be flipped in or flipped out and not revealed during fitting, possibly resulting in leakage during wear.

Thus, there is a need for a disposable diaper including features that facilitate the changing process. Particularly, there is a need for a diaper having intuitive elements that facilitate a change by providing a caregiver an apparent visual and physical indication when the article is not properly fitted to the wearer.

### SUMMARY

The present invention provides a disposable absorbent article worn about the lower torso of a wearer that includes at least one serviceable indicium that facilitates the process of fitting the absorbent article to the wearer by providing an apparent visual and physical indication when the article is not properly fitted. The apparent visual and physical indication includes a detectable distortion of the waist opening, the leg openings or both the waist opening and the leg openings. The detectable distortion includes a portion of the waist opening or leg opening or both either flipped in towards an inner surface of the article or flipped out towards the outer surface of the article. By detectable it is meant that the distortion is large enough to be visually apparent to the caregiver.

Embodiments of the disposable absorbent article include a disposable absorbent article including a body facing surface and a garment facing surface; a first waist region with a first end edge, a second waist region with a second end edge, and a crotch region interposed between the first waist region and the second waist region. The first end edge and second end edge are joined by opposing longitudinal side edges. The disposable absorbent article comprises a backsheet, a topsheet disposed on the backsheet and a core interposed between the topsheet and backsheet. Serviceable indicia are disposed on portions of the body facing surface of the article, the garment facing surface, both the body facing and the garment facing surface or between the body facing surface and garment facing surface of the article. The serviceable indicia are located proximate at least portions of the opposing longitudinal side edges, proximate at least portions of the first end edge or the second end edge, or proximate at least portions of both the opposing longitudinal side edges and proximate at least portions of the first end edge and the second end edge in the first and second waist regions, respectively. The portions of the opposing longitudinal side edges and/or portions of the first and second end edges including the serviceable indicia have a buckling force which is greater than the buckling force of portions of the article inboard of and adjacent to the serviceable indicia. Particularly, the portions of the first and second end edges including the serviceable indicia have a buckling force which is greater than 1.8 N and the portions of the opposing longitudinal side edges including the serviceable indicia have a buckling force which is greater than 1.0 N. In addition, the ratio of the buckling force of the portion of the article including the serviceable indicia to the buckling force of the portions of the article inboard of and adjacent to the serviceable indicia is at least about 4.0.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIG. 1 is a plan view of a disposable diaper constructed in accordance with the present invention having portions cut away to reveal underlying structure. The body-facing surface of the diaper is facing the viewer.

FIG. 2 is a plan view of the garment facing surface of the disposable diaper in FIG. 1.

FIG. 3 is an isometric view of the diaper in FIG. 1 configured in a fitted orientation.

FIG. 4 is an isometric view of a pant style diaper constructed in accordance with the present invention.

FIG. 5 is a general setup of a constant rate of extension tensile tester used to evaluate buckling force.

FIG. 6 is illustrates a test sample of a disposable absorbent including serviceable indicia in accordance with the present invention tested to determine buckling force.

FIG. 7 is a stress v. strain curve used to calculate buckling force.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and study of the included drawings.

### DEFINITIONS

As used herein, the following terms have the following meanings:

“Absorbent article” refers to devices that absorb and contain liquid, and more specifically, refers to devices that are placed against or in proximity to the body of the wearer to absorb and contain the various exudates discharged from the body.

“Longitudinal” is a direction running parallel to the maximum linear dimension of the article and includes directions within ±45° of the longitudinal direction.

The “lateral” or “transverse” direction is orthogonal to the longitudinal direction and is essentially in the plane of the article when the article is in a flat stretched out position.

The “Z-direction” is orthogonal to both the longitudinal and transverse directions.

The “x-y plane refers to the plane congruent with the longitudinal and transverse directions.

The term “disposable” is used herein to describe absorbent articles that generally are not intended to be laundered or otherwise restored or reused as an absorbent article (i.e., they are intended to be discarded after a single use and, preferably, to be recycled, composted or otherwise disposed of in an environmentally compatible manner).

As used herein, the term “disposed” is used to mean that an element(s) is formed (joined and positioned) in a particular place or position as a unitary structure with other elements or as a separate element joined to another element.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

A “unitary” absorbent article refers to absorbent articles which are formed of separate parts united together to form a coordinated entity so that they do not require separate manipulative parts like a separate holder and liner.

As used herein, the term “complement” refers to filling in or completing such as by overlapping, matching, or aligning therewith; contextually relating, or highlighting.

As used herein, the term “body-facing surface” generally refers to a surface oriented towards the body when fitted to a wearer.

As used herein, the term “garment-facing surface” generally refers to a surface oriented opposite the body-facing surface when fitted to a wearer.

As used herein, the term “serviceable indicium or indicia” generally refers to distinctive marks, colored regions, patterns, and/or textures disposed on a disposable absorbent article to provide a functional attribute. Particularly, the functional attribute includes providing a detectable distortion indicating that article is not properly fitted about the wearer.

As used herein the term detectable distortion means a large enough portion of an article or material is deformed to be externally visible and distinctly discernible.

As used herein, the term “externally visible”, as used in reference to an indicium associated with an article, refers to the ability of a human viewer to visually discern the indicium with the unaided eye (excepting standard corrective lenses adapted to compensate for near-sightedness, farsightedness, or astigmatism) in standard lighting conditions from a point of reference viewing the garment-facing surface of the article while the article is fitted to a wearer wherein the garment-facing surface is within the field of view.

As used herein, “standard lighting conditions” refer to lighting conditions in which human vision operates efficiently (e.g., the human eye is able to discern complex patterns, shading, and colors). Specifically, for the purposes of describing this invention, standard lighting conditions are at least one of the following:
a) natural illumination as experienced outdoors during daylight hours,
b) the illumination of a standard 100 watt incandescent white light bulb at a distance of 2 meters, or
c) as defined by CIE D65 standard illuminate lighting at 800 lux to a 1964 CIE standard observer.

As used herein, an “anatomic feature” of a wearer may include any externally discernible portion of the wearer's anatomy specific to a certain definable region and/or function. Exemplary anatomic features of human bodies include, without limitation, waste exit ports such as the anus, genitalia, the perineal region, the gluteal groove, leg creases, the navel, buttocks, hip and/or pubic bones, the thighs, the rib cage, and the like.

The terms “permeable” and “impermeable” refer to the penetrability of materials in the context of the intended usage of disposable absorbent articles. Specifically, the term “permeable” refers to a layer or a layered structure having pores or openings that permit liquid water to pass through its thickness in the absence of a forcing pressure. Conversely, the term “impermeable” generally refers to articles and/or elements that are not penetrative by fluid through the entire Z-directional thickness of the article under pressure of 0.14 lb/in2 or less. Preferably, the impermeable article or element is not penetrative by fluid under pressures of 0.5 lb/in2 or less. More preferably, the impermeable article or element is not penetrative by fluid under pressures of 1.0 lb/in2 or less.

The term “attached” refers to elements being connected or united by fastening, adhering, bonding, etc. by any method suitable for the elements being fastened, secured, or joined, together and their constituent materials. Many suitable methods for attaching elements together are well-known, including adhesive bonding, pressure bonding, thermal bonding, mechanical fastening, etc. Such attachment methods may be used to attach elements together over a particular area either continuously or intermittently. The term “attached” includes elements which are integrally formed with another element.

The terms “corrugations” or “rugosities” are used to describe hills and valleys that occur in a substrate or in a laminated structure. Neither term, i.e. “corrugations” nor “rugosities”, mandates that either the hills or valleys created are uniform in nature.

As used herein, the term “diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

The terms “pant”, “pant style diaper”, “training pant”, “closed diaper”, “pre-fastened diaper”, and “pull-on diaper”, as used herein, refer to disposable garments having a waist opening and leg openings designed for infant or adult wearers. A pant can be configured such that the pant has a closed waist and leg openings prior to being donned on the wearer or the pant can be configured such that the waist is closed and the leg openings are formed while on the wearer. A pant may be preformed by any suitable technique including, but not limited to, attaching together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened, rear waist fastened). Examples of suitable pants are disclosed in U.S. Pat. No. 5,246,433; U.S. Pat. No. 5,569,234; U.S. Pat. No. 6,120,487; U.S. Pat. No. 6,120,489; U.S. Pat. No. 4,940,464; U.S. Pat. No. 5,092,861; U.S. Pat. No. 5,897,545; U.S. Pat. No. 5,957,908; and U.S. Patent Publication No. 2003/0233082 A1.

As used herein “elastically extensible” refers to characteristics of extensible materials that have the ability to return to approximately their original dimensions after a force that extended the extensible material is removed. Herein, any material or element described as “extensible” may also be “elastically extensible” unless otherwise provided.

As used herein the term “stretch” means to forcibly extend in length or width.

The term “live stretch” includes stretching elastic and bonding the stretched elastic to a nonwoven. After bonding the stretched elastic is released causing it to contract, resulting in a “corrugated” nonwoven. The corrugated nonwoven can stretch as the corrugated portion is pulled to about the point that the nonwoven reaches at least one original flat dimension. The elastic is preferably stretched at least 25% and more preferably at least 100% of its relaxed length when it is bonded to the nonwoven.

As used herein the term “inboard of” refers to a direction which is towards a point of intersection between longitudinal and transverse axis.

Buckling force means a force at which a material will begin to bend when subject to collinear compressive forces in excess of the yield strength (elastic limit) of the material.

### DESCRIPTION

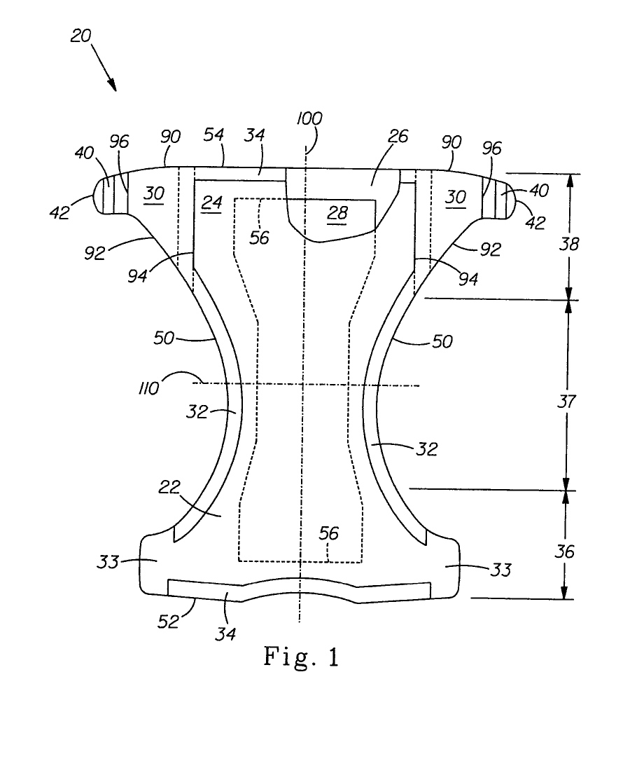

The present invention provides a disposable absorbent article worn about the lower torso of a wearer including at least one serviceable indicium that facilitates changing the article on a wearer. The serviceable indicium is disposed in distinct areas of the disposable absorbent article and provides an apparent visual and physical indication when the article is not properly fitted to the wearer. The apparent visual and physical indication comprises a detectable distortion where the portion of the article including the serviceable indicium is flipped in or flipped out. The wearable article may be applicable to disposable absorbent articles including training pants, incontinence briefs, incontinence undergarments, inserts for disposable or durable diapers or other garments and the like. One embodiment of an absorbent article of the present invention is a unitary disposable absorbent article, such as the disposable diaper 20, shown in FIG. 1.

FIG. 1 is a plan view of the diaper 20 of the present invention in a flat-out state with portions of the structure being cut-away to more clearly show the construction of the diaper 20. The portion of the diaper 20 which faces the wearer is oriented towards the viewer. The diaper 20 has a first waist region 36, a second waist region 38 opposed to the first waist region 36 and a crotch region 37 located between the first waist region 36 and the second waist region 38. The periphery of the diaper 20 is defined by the outer edges of the diaper 20 in which opposing longitudinal side edges 50 run generally parallel to the longitudinal centerline 100 of the diaper 20 and a first end edge in the first waist region and a second end edge 52 in the second waist region. The first and second end edges run between the opposing longitudinal side edges 50 generally parallel to the lateral centerline 110 of the diaper 20.

As shown in FIG. 1, the diaper 20 comprises a liquid pervious topsheet 24; a liquid impervious backsheet 26; an absorbent core 28 which is preferably positioned between at least a portion of the topsheet 24 and the backsheet 26; side panels 30; extensible leg cuffs 32, elastic waist features 34 and a fastening system generally designated 40. The chassis 22 of the diaper 20 comprises the main body of the diaper 20 and includes the topsheet 24 and/or the backsheet 26 and at least a portion of the absorbent core 28. While the topsheet 24, the backsheet 26, the absorbent core 28, side panels 30 and other aforementioned constituents may be assembled in a variety of well known configurations, preferred diaper configurations are described generally in U.S. Pat. No. 3,860,003 entitled “Contractible Side Portions for Disposable Diaper” issued to Kenneth B. Buell on Jan. 14, 1975; U.S. Pat. No. 5,151,092 issued to Buell on Sep. 9, 1992; and U.S. Pat. No. 5,221,274 issued to Buell on Jun. 22, 1993; and U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; U.S. Pat. No. 5,580,411 entitled “Zero Scrap Method for Manufacturing Side Panels for Absorbent Articles” issued to Nease et al. on Dec. 3, 1996; and U.S. Pat. No. 6,004,306 entitled “Absorbent Article With Multi-Directional Extensible Side Panels” issued to Robles et al. on Dec. 21, 1999.

The side panels 30 can have a number of different sizes and shapes, but preferably have a trapezoidal shape. The side panels 30 can be elastic or extensible to provide a more comfortable and contouring fit by initially conformably fitting the diaper 20 to the wearer and sustaining this fit throughout the time of wear. Side panels 30 which are elasticized can provide a sustained fit well past when the diaper 20 has been loaded with exudates since the elasticized side panels 30 allow the sides of the diaper 20 to expand and contract. The side panels 30 may also provide more effective application of the diaper 20 because even if one elasticized side panel 30 is pulled farther than the other during application, the diaper 20 will “self-adjust” during wear.

The side panels 30 may comprise a separate element affixed to the chassis 22, or can be constructed as an extension of other elements of the diaper such as the backsheet 26 or the topsheet 24, preferably both the topsheet 24 and the backsheet 26. In the embodiment shown in FIG. 1, the side panels 30 each comprise a separate web joined to the chassis 22 in the second waist region 38 and extend laterally outwardly beyond the longitudinal edges 50. The side panels 30 comprise a waist edge 90 positioned adjacent the first end edge 52 of the diaper, a leg edge 92 positioned away from the first end edge 90 towards the lateral centerline 110, a first longitudinal edge 94 attached to the longitudinal edge 50, and a second longitudinal edge 96 positioned laterally outwardly from the longitudinal edge 50. The first longitudinal edge 94 may be contiguous with the longitudinal edge 50, preferably the first longitudinal edge 94 is positioned laterally inwardly of the longitudinal edge 50. The side panels 30 may include a nonelastic extension members 42 attached to the second longitudinal edges 96 and fasteners 40 attached to the nonelastic extension members 42. The nonelastic extension members 42 can be made to distribute the shear forces along the second longitudinal edges 96 of the side panels 30 during fastening.

The diaper 20 may be provided with side panels 30 disposed in the first waist region 36 or in both the first waist region 36 and the second waist region 38. As shown in FIG. 1, the diaper 20 can also include side panels, referred to as second side panels 33, disposed in the first waist region 36. Similar to the side panels 30, the second side panels 33 may be integral with the chassis 22 comprising extensions of a unibody chassis design or alternatively, the second side panels 33 may comprise separate members attached to the chassis 22 using adhesives, ultrasonic bonds, radio frequency bonds, or other suitable means.

The side panels 30, 33 may be constructed in any suitable configuration accommodating a particular product design. Examples of diapers with elasticized side panels are disclosed in U.S. Pat. No. 4,857,067, entitled “Disposable Diaper Having Shirred Ears” issued to Wood, et al. on Aug. 15, 1989; U.S. Pat. No. 4,381,781 issued to Sciaraffa, et al. on May 3, 1983; U.S. Pat. No. 4,938,753 issued to Van Gompel, et al. on Jul. 3, 1990; the herein before referenced U.S. Pat. No. 5,151,092 issued to Buell on Sep. 9, 1992; U.S. Pat. No. 5,221,274 issued to Buell on Jun. 22, 1993; U.S. Pat. No. 5,669,897 issued to LaVon, et al. on Sep. 23, 1997 entitled “Absorbent Articles Providing Sustained Dynamic Fit”; and U.S. Pat. No. 6,004,306 entitled “Absorbent Article With Multi-Directional Extensible Side Panels” issued to Robles et al. on Dec. 21, 1999.

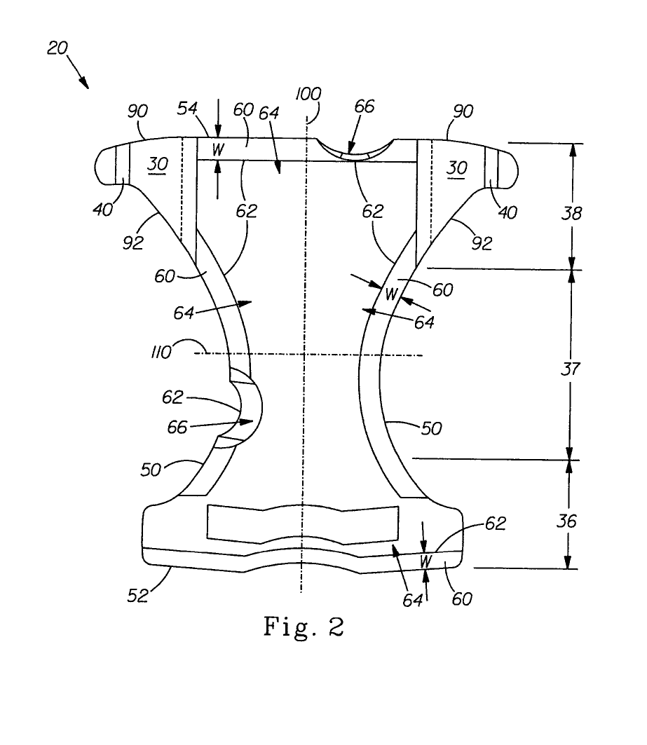

An exemplary disposable absorbent article including serviceable indicia 60 according to the present invention is the disposable diaper 20 shown in FIG. 2 with the garment facing surface of the diaper 20 facing the viewer. The disposable diaper 20 includes at least one serviceable indicium 60 disposed along at least portions of the article proximate the longitudinal side edges 50 and/or at least one serviceable indicium 60 disposed along portions of the article proximate the first end edge 52 and/or second end edge 54 in the first waist region 36 and second waist regions 38, respectively. The portions of the article including the serviceable indicia 60 are physically discernable such that the buckling force across the width of the indicia is both sufficient to resist a bending distortion itself and exceeds the buckling force of portions of the article inboard of and adjacent to the serviceable indicia 60, enabling the indicium to signal improper application of the article to the wearer. The serviceable indicia 60 include outer edges contiguous with the longitudinal side edges 50 and first end edge 52 and second end edge 54 and inner edges forming a hinge point 62 between the serviceable indicia 60 and the portion 64 of the article inboard of and adjacent to the serviceable indicia 60. The serviceable indicia 60 can deflect about the hinge point 62 producing a detectable distortion 66 signaling improper application to the wearer.

In addition to providing the aforementioned functional attribute, the serviceable indicia 60 may also include a color, texture and/or a pattern distinguishing the serviceable indicia 60 from other portion of the article.

For serviceable indicia 60 comprising a pattern, the pattern may be in the form of a series of shapes and/or images. For example, the pattern may be formed of one or more dots, one or more lines, one or more regular or irregular shapes (such as circles, ellipses, diamonds, squares, and the like), or combinations thereof. Alternatively, a pattern may be in the form of a variation of color along a length of the serviceable indicia 60. For example, the color may vary from light to dark or from one hue to another. Images may include drawings of characters or objects readily recognizable to children.

The serviceable indicia 60 may be disposed directly on the garment-facing surface of the backsheet 26, on the surface of the backsheet 26 opposite the garment-facing surface adjacent to the core 28, on one of the components of the backsheet 26, or beneath the backsheet 26 on underlying layers.

The serviceable indicia 60 generally include a structure that differs from other portions of the article and that exhibits a buckling force sufficient to cause a physically discernable distortion as a signal of improper fit and which exceeds the buckling force of portions 64 of the article inboard of and adjacent to the serviceable indicia 60. It is important that the buckling force of the serviceable indicium 60 exceed a threshold value so as to prevent the indicium 60 itself from bending when improperly applied to the wearer. In other words the serviceable indicium 60 includes a structure that is not prone to bending (threshold buckling force) where as the region inboard of and adjacent to the indicium is prone to bending (low buckling force). The physically discernable distortion should occur immediately inboard of the indicium for maximum effect. Therefore, the transition between the serviceable indicia 60 and the portions inboard of and adjacent thereto forms the hinge point 62 enabling the serviceable indicia 60 to provide a detectable distortion 66 shown in FIG. 2. For instance, the serviceable indicia 60 are predisposed to bend at the hinge point 62 and to fold completely in towards the body facing surface or out towards the garment facing surface if the article is improperly fitted. The structural differences can include an additional layer of material or a laminate attached to the article. Alternatively, the structural difference may comprise a texture formed by mechanical treatment and or thermal treatment.

For serviceable indicia 60 comprising texture, portions of the diaper 20 along the first and second end edges and the longitudinal side edges 50 may be mechanically treated to provide texture by methods known in the art, including mechanical operations, such as pleating, corrugating, or ring rolling to provide rugosities generally orthogonal to the first and second end edges and the longitudinal side edges. In addition to providing texture which increases the stiffness and corresponding buckling force of the region being worked, these mechanical operations may also provide extensibility. Suitable processes for mechanical treatment such as ring rolling or pre-corrugating, including extensible webs made thereby, are described in U.S. Pat. No. 4,107,364 issued to Sisson on Aug. 15, 1978; U.S. Pat. No. 4,834,741 issued to Sabee on May 30, 1989; U.S. Pat. No. 5,167,897 issued to Weber et al. on Dec. 1, 1992, and U.S. Pat. No. 5,702,382 issued to Osborn, III et al. on Dec. 30, 1997.

Alternatively, serviceable indicia 60 comprising texture may be formed by forming a strainable network having at least two contiguous, distinct, and dissimilar regions. One of the regions can include rib-like elements oriented to enhance the buckling force. Films thus formed have in the past been termed structural elastic-like films (“SELF”). A structural elastic-like film or web is an extensible material that can exhibit an elastic-like behavior in the direction of elongation without the use of added elastic materials. However, SELF webs can be made which exhibit little elastic behavior. In particular, webs comprising a laminate of films and nonwovens can be made which exhibit little elastic behavior beyond very low levels of strainch

Serviceable indicia 60 comprising SELF suitable for the present invention, and methods of forming SELF webs, are more completely described in U.S. Pat. No. 5,518,801 entitled Web Materials Exhibiting Elastic-Like Behavior, issued to Chappell, et, al. on May 21, 1996; U.S. Pat. No. 5,650,214 entitled Web Materials Exhibiting Elastic-Like Behavior and Soft, Cloth-like Texture, issued to Anderson et al. on Jul. 22, 1997; and U.S. Pat. No. 5,904,673 entitled Absorbent Article with Structural Elastic-like Film Web Waist Belt, issued to Roe et al. on May 18, 1999, all of which are hereby incorporated herein by reference. For film/nonwoven laminate backsheets, the processes described in the above-mentioned patents can be performed on the laminate material or on the separate components prior to lamination, or both.

In certain embodiments, the serviceable indicia 60 may be sufficiently opaque, or have a sufficiently dark color, to additionally provide a masking benefit, effectively preventing visual detection of a layer, material, or substance underlying the serviceable indicia 60. For example, the serviceable indicia 60 proximate the longitudinal side edges 14 of a diaper 20 may have opacity such that feces and/or portions of the absorbent core 28 that may be present under the region of the backsheet 26 proximate the longitudinal side edges 14 of the diaper 20 are not visible from outside the diaper 20.

In addition to providing a detectable distortion 66, the serviceable indicia 60 may provide other functional attributes such as influencing how the diaper fits on a wearer. For the embodiment illustrated in FIG. 2, the serviceable indicia 60 disposed about the first and second end edges can include the elastic waist feature 34 while the serviceable indicia 60 about the opposing longitudinal side edges can include the extensible leg cuffs.

Elastic waist feature 34 helps to provide improved fit and containment. The elastic waist feature 34 is generally intended to elastically expand and contract to dynamically fit the wearer's waist. The elastic waist feature 34 preferably extends at least longitudinally outwardly from at least one waist edge 56 of the absorbent core 28 and generally forms at least a portion of the end edge 52 of the diaper 20. Disposable diapers are often constructed so as to have two elastic waist features, one positioned in the first waist region 36 and one positioned in the second waist region 38.

The elastic waist feature 34 or any of its constituent elements may comprise one or more separate elements affixed to the diaper 20. The elastic waist feature 34 can be constructed to function as serviceable indicia 60 according to the present invention by including a structure with the necessary buckling force to produce a detectable distortion 66 if the diaper is not properly fitted about the wearer's waist. The elastic waist feature 34 can be constructed with conventional elastic materials or stretch laminates. The stretch laminates may comprise a laminated structure known as live stretch, previously defined, where an elastic element is attached to a substrate while the elastic element is under strain; such that once the strain is relieved the laminate forms corrugations or gathers and exhibits a shined structure having elastic-like properties. The corrugations or gathers provide stiffness across the width of the stretch laminate resulting in an elastic waist feature that exhibits requisite buckling force once attached proximate the first and second end edges of the diaper. The elastic element may include an elastomer extruded onto the nonwoven or a film that is glued to the nonwoven. Alternatively, the elastic waist feature 34 may comprise a laminate structure comprising two layers of nonwoven with elastomeric strands or a film disposed therebetween covering the full width of the laminate or only a portion of the width.

Alternatively, the stretch laminate may comprise a mechanically activated stretched laminate such as a zero strain stretch laminate. Zero strain stretch laminates comprise a laminated structure which includes a first substrate, a second substrate and an elastic element. The first substrate and the second substrate, which are typically non-elastic nonwovens, are attached to the elastic element in a face to face orientation such that the elastic element is sandwiched between the first substrate and the second substrate. The laminated structure is mechanically activated enabling it to stretch. Mechanical activation refers to a process wherein the nonwoven fibers of the non-elastic substrates are broken, and/or stretched, within the nonwoven so that the nonwoven is stretched in a direction along its surfaces and can be easily expanded in that direction by partial straightening of the fibers in the nonwoven. Mechanical activation also forms ribs in the laminate which are perpendicular to the direction of stretch. The ribs can provide stiffness across the width of the stretch laminate enabling the elastic waist feature to exhibit the requisite buckling force. Zero-strain elastomeric laminates are described in U.S. Pat. No. 5,143,679 issued to Weber et al., U.S. Pat. No. 5,156,793 issued to Buell et al., and U.S. Pat. No. 5,167,897 issued to Weber.

The elastic waist feature 34 may be attached to the outer, garment facing surface of the backsheet; the body facing surface of the topsheet or both. In addition the elastic waist feature may be attached between the topsheet and the backsheet, or wrapped around the end edges 52 of the diaper 20 and attached to both the body-facing surface of the topsheet and the garment-facing surface of the backsheet.

The diaper 20 can also comprise extensible leg cuffs 32 for providing improved containment of liquids and other body exudates. Each extensible leg cuff 32 may comprise several different embodiments for reducing the leakage of body exudates in the leg regions. (The leg cuff can be and is sometimes also referred to as leg bands, leg flaps, barrier cuffs, or elastic cuffs.) The extensible leg cuffs 32 may be attached to the outer, garment facing surface of the backsheet; the body facing surface of the topsheet or both. In addition, the extensible leg cuffs 32 may be attached between the topsheet and the backsheet, or wrapped around the longitudinal side edges 50 of the diaper 20 and attached to both the body-facing surface of the topsheet and the garment-facing surface of the backsheet.

Like the elastic waist feature 34, the extensible leg cuffs 32 may be constructed in a number of different configurations influencing not only how the diaper fits about the wearer but also to function as serviceable indicia 60 according to the present invention by constructing the extensible leg cuffs with the necessary buckling force to produce a detectable distortion 66 if the diaper is not properly fitted about the wearer's legs. For instance, the extensible leg cuffs 32 may comprise a laminate structure consisting of a single layer of nonwoven with an elastomeric material attached covering a full width dimension of the nonwoven or only a portion of the width. The elastomeric material may include an extruded elastic strand laminate comprising prestrained extruded elastic strands laminated to a nonwoven or a film that is subsequently glued to the garment facing surface of the backsheet along the longitudinal edges 50 of the diaper 20 and allowed to relax to form a shirred or corrugated structure. Alternatively, the extensible leg cuffs 32 may comprise a laminate structure comprising two layers of nonwoven with prestrained extruded elastic strands laminated between the two layers of nonwoven covering the full width of the laminate or only a portion of the width. Alternatively, the laminate structure may comprise a mechanically activated stretch laminate such as a zero strain stretch laminate. The laminate structure can be subsequently glued to the garment facing surface of the backsheet along the longitudinal edges 50 of the diaper 20 while in the prestrained condition so that a shined or corrugated structure is formed once tension is removed from the laminate allowing it to relax.

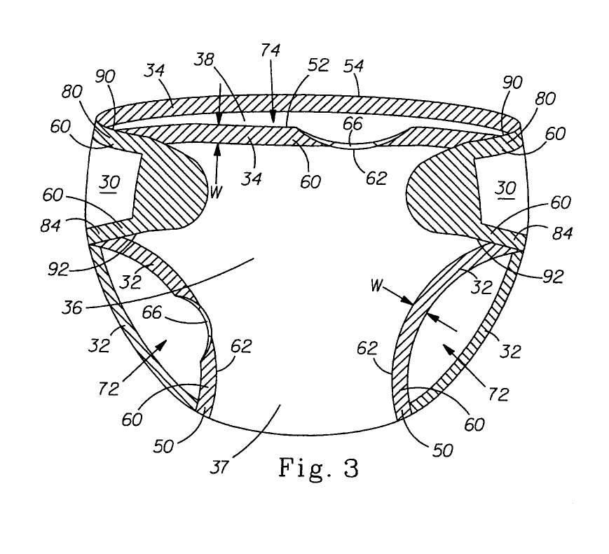

FIG. 3 depicts a diaper 20 in a fastened configuration illustrating how the serviceable indicia 60 provide a detectable distortion 66 about the leg opening 72 and waist opening 74 indicating improper fit. In addition, FIG. 3 illustrates how the serviceable indicia 60 provide other functional attributes such as provide an alignment guide enabling the caregiver to properly align the diaper on the wearer during fitting. As shown, each side panel 30 includes a waist region 80 proximate waist edge 92 and a leg region 84 proximate leg edge 92 with serviceable indicia 60 in the waist region 80 and the leg region 84. The serviceable indicia 60 in the leg region 84 of the side panel 30 can be made to match the serviceable indicia 60 proximate the longitudinal edges 50 and the serviceable indicia 60 in the waist region 80 can be made to match the serviceable indicia 60 proximate end edges 52 in the first and second waist regions 36, 38. Once the diaper 20 is donned on a wearer, the waist regions 80 of the side panels 30 align with the end edges 52 in the first and second waist regions 36, 38 forming a complete 360° waist band encircling the waist opening 74 of the diaper 20. At the same time, the leg regions 84 of the side panels 30 align with the longitudinal edges 50 of the diaper to form 360° leg bands encircling the leg openings 72.

In addition to visual distinctions, the serviceable indicia 60 can provide additional functional attributes. For instance, the elastic waist feature 34 in the second waist region 38 can be made to extend into one or both of the side panels 30 partially or fully covering the waist region 80 in one or both of the side panels 30. For the embodiment shown in FIG. 3, the elastic waist feature 34 in the second waist region 38 is shown fully covering the waist region 80 in each of the side panels 30 such that the combination of the elastic waist feature 34 in the second waist region 38 of the diaper and the waist region 80 in each of the side panels 30 aligns with the elastic waist feature 34 in the first waist region 36 of the diaper forming a 360° elastic waist about the waist opening 74 when the diaper 20 is donned on a wearer.

Similarly, the extensible leg cuffs 32 can be made to extend into the leg region 84 of one or both of the side panels 30 partially or fully covering the leg region 84 in one or both of the side panels 30. As shown in FIG. 3, the extensible leg cuffs 32 can be made to fully cover the leg region 84 in each of the side panels 30 such that the combination of the leg region 84 in each of the side panels 30 and the extensible leg cuffs 32 form complete 360° leg elastics about each of the leg openings 72. For this embodiment, the extensible leg cuffs 32 and the leg regions 84 in each of the side panels can include aforementioned visual features to appear as garment-like seams about the leg openings 72.

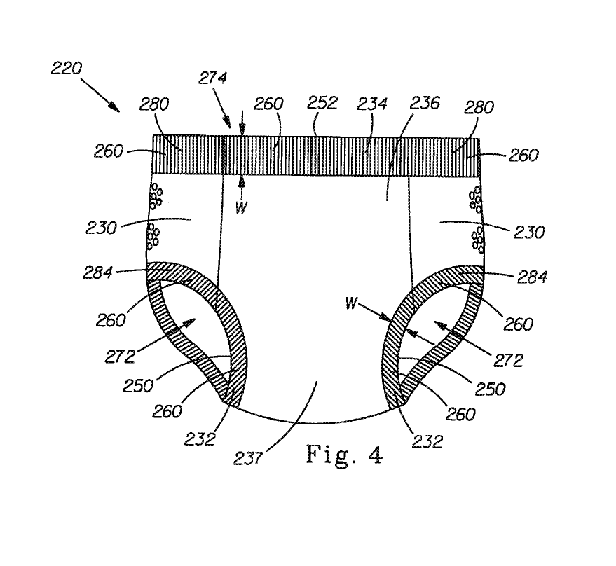

FIG. 4 depicts a pant style diaper 220 illustrating how the serviceable indicia 260 can be made to enhance fit as well as a garment-like look. As shown, the serviceable indicia 260 in the waist regions 280 of the side panels 230 comprise a continuation of serviceable indicia 260 proximate the end edges 252 in the first waist region 236. The serviceable Indicia 260 proximate end edges 252 can include an elastic waist feature 234 extending into, and partially or fully covering the waist region 280 in each of the side panels 230. Although not shown, the waist regions 280 of the side panels 230 can also comprise a continuation of a serviceable indicia 260 and/or elastic waist feature proximate the end edges 252 in the second waist regions forming, in combination with the elastic waist feature 234 in the first waist region 236, a 360° waist band encircling the waist opening 274. In addition, as shown in FIG. 4, serviceable indicia 260 proximate the leg regions 284 of the side panels 230 can comprise a continuation of serviceable indicia 260 proximate the longitudinal edges 250 in the crotch region 237 of the pant style diaper 220 forming 360° leg bands encircling the leg openings 272. The serviceable indicia 260 in the leg regions 284 may also include extensible leg cuffs 232 partially or fully covering the leg regions 284 in each of the side panels 230.

Other components of the chassis 22 include the backsheet 26, the topsheet 24 and the core 28. The backsheet 26 is generally that portion of the diaper 20 positioned adjacent garment facing surface of the absorbent core 28 which prevents the exudates absorbed and contained therein from soiling articles which may contact the diaper 20, such as bed sheets and undergarments. In preferred embodiments, the backsheet 26 is impervious to liquids (e.g., urine) and comprises a thin plastic film such as a thermoplastic film having a thickness of about 0.012 mm (0.5 mil) to about 0.051 mm (2.0 mils). Suitable backsheet films include those manufactured by Tredegar Corporation, based in Richmond, Va., and sold under the trade name CPC2 film. Other suitable backsheet materials may include breathable materials which permit vapors to escape from the diaper 20 while still preventing exudates from passing through the backsheet 26. Exemplary breathable materials may include materials such as woven webs, nonwoven webs, composite materials such as film-coated nonwoven webs, microporous films such as manufactured by Mitsui Toatsu Co., of Japan under the designation ESPOIR NO and by Tredegar Corporation of Richmond, Va. and sold under the designation EXAIRE., and monolithic films such as manufactured by Clopay Corporation, Cincinnati, Ohio under the name HYTREL blend P18-3097. Some breathable composite materials are described in greater detail in PCT Application No. WO 95/16746 published on Jun. 22, 1995 in the name of E. I. DuPont; U.S. Pat. No. 5,938,648 issued on Aug. 17, 1999 to LaVon et al.; U.S. Pat. No. 5,865,823 issued on Feb. 2, 1999 in the name of Curro; and U.S. Pat. No. 5,571,096 issued to Dobrin et al. on Nov. 5, 1996.

The backsheet 26 may be joined to the topsheet 24, the absorbent core 28 or any other element of the diaper 20 by any attachment means known in the art. For example, the attachment means may include a uniform continuous layer of adhesive, a patterned layer of adhesive, or an array of separate lines, spirals, or spots of adhesive. One preferred attachment means comprises an open pattern network of filaments of adhesive as disclosed in U.S. Pat. No. 4,573,986 entitled “Disposable Waste-Containment Garment”, which issued to Minetola et al. on Mar. 4, 1986. Other suitable attachment means include several lines of adhesive filaments which are swirled into a spiral pattern, as is illustrated by the apparatus and methods shown in U.S. Pat. No. 3,911,173 issued to Sprague, Jr. on Oct. 7, 1975; U.S. Pat. No. 4,785,996 issued to Ziecker, et al. on Nov. 22, 1978; and U.S. Pat. No. 4,842,666 issued to Werenicz on Jun. 27, 1989. Adhesives which have been found to be satisfactory are manufactured by H. B. Fuller Company of St. Paul, Minn. and marketed as HL-1620 and HL 1358-XZP. Alternatively, the attachment means may comprise heat bonds, pressure bonds, ultrasonic bonds, dynamic mechanical bonds, or any other suitable attachment means or combinations of these attachment means as are known in the art.

The topsheet 24 is preferably positioned adjacent body surface of the absorbent core 28 and may be joined thereto and/or to the backsheet 26 by any attachment means known in the art. Suitable attachment means are described above with respect to means for joining the backsheet 26 to other elements of the diaper 20. In one preferred embodiment of the present invention, the topsheet 24 and the backsheet 26 are joined directly to each other in some locations and are indirectly joined together in other locations by directly joining them to one or more other elements of the diaper 20.

The topsheet 24 is preferably compliant, soft-feeling, and non-irritating to the wearer's skinch Further, at least a portion of the topsheet 24 is liquid pervious, permitting liquids to readily penetrate through its thickness. A suitable topsheet may be manufactured from a wide range of materials, such as porous foams, reticulated foams, apertured plastic films, or woven or nonwoven materials of natural fibers (e.g., wood or cotton fibers), synthetic fibers (e.g., polyester or polypropylene fibers), or a combination of natural and synthetic fibers. If the topsheet 24 includes fibers, the fibers may be spunbond, carded, wet-laid, meltblown, hydroentangled, or otherwise processed as is known in the art. One suitable topsheet 24 comprising a web of staple-length polypropylene fibers is manufactured by Veratec, Inc., a Division of International Paper Company, of Walpole, Mass. under the designation P-8.

Suitable formed film topsheets are described in U.S. Pat. No. 3,929,135, entitled “Absorptive Structures Having Tapered Capillaries” issued to Thompson on Dec. 30, 1975; U.S. Pat. No. 4,324,246 entitled “Disposable Absorbent Article Having A Stain Resistant Topsheet” issued to Mullane, et al. on Apr. 13, 1982; U.S. Pat. No. 4,342,314 entitled “Resilient Plastic Web Exhibiting Fiber-Like Properties” issued to Radel, et al. on Aug. 3, 1982; U.S. Pat. No. 4,463,045 entitled “Macroscopically Expanded Three-Dimensional Plastic Web Exhibiting Non-Glossy Visible Surface and Cloth-Like Tactile Impression” issued to Ahr, et al. on Jul. 31, 1984; and U.S. Pat. No. 5,006,394 “Multilayer Polymeric Film” issued to Baird on Apr. 9, 1991. Other suitable topsheets may be made in accordance with U.S. Pat. Nos. 4,609,518 and 4,629,643 issued to Curro et al. on Sep. 2, 1986 and Dec. 16, 1986, respectively. Such formed films are available from The Procter & Gamble Company of Cincinnati, Ohio as “DRI-WEAVE” and from Tredegar Corporation, based in Richmond, Va., as “CLIFF-T.”

The absorbent core 28 may comprise any absorbent material which is generally compressible, conformable, non-irritating to the wearer's skin, and capable of absorbing and retaining liquids such as urine and other certain body exudates. The absorbent core 28 may be manufactured in a wide variety of sizes and shapes (e.g., rectangular, hourglass, “T”-shaped, asymmetric, etc.) and may comprise a wide variety of liquid-absorbent materials commonly used in disposable diapers and other absorbent articles such as comminuted wood pulp, which is generally referred to as airfelt. Examples of other suitable absorbent materials include creped cellulose wadding; meltblown polymers, including coform; chemically stiffened, modified or cross-linked cellulosic fibers; tissue, including tissue wraps and tissue laminates; absorbent foams; absorbent sponges; superabsorbent polymers; absorbent gelling materials; or any other known absorbent material or combinations of materials.

The configuration and construction of the absorbent core 28 may also be varied (e.g., the absorbent core(s) or other absorbent structure(s) may have varying caliper zones, hydrophilic gradient(s), a superabsorbent gradient(s), or lower average density and lower average basis weight acquisition zones; or may comprise one or more layers or structures).

Exemplary absorbent structures for use as the absorbent core 28 are described in U.S. Pat. No. 4,610,678 entitled “High-Density Absorbent Structures” issued to Weisman et al. on Sep. 9, 1986; U.S. Pat. No. 4,673,402 entitled “Absorbent Articles With Dual-Layered Cores” issued to Weisman et al. on Jun. 16, 1987; U.S. Pat. No. 4,834,735 entitled “High Density Absorbent Members Having Lower Density and Lower Basis Weight Acquisition Zones” issued to Alemany et al. on May 30, 1989; U.S. Pat. No. 4,888,231 entitled “Absorbent Core Having A Dusting Layer” issued to Angstadt on Dec. 19, 1989; U.S. Pat. No. 5,137,537 entitled “Absorbent Structure Containing Individualized, Polycarboxylic Acid Crosslinked Wood Pulp Cellulose Fibers” issued to Herron et al. on Aug. 11, 1992; U.S. Pat. No. 5,147,345 entitled “High Efficiency Absorbent Articles For Incontinence Management” issued to Young et al. on Sep. 15, 1992; U.S. Pat. No. 5,342,338 entitled “Disposable Absorbent Article For Low-Viscosity Fecal Material” issued to Roe on Aug. 30, 1994; U.S. Pat. No. 5,260,345 entitled “Absorbent Foam Materials For Aqueous Body Fluids and Absorbent Articles Containing Such Materials” issued to DesMarais et al. on Nov. 9, 1993; U.S. Pat. No. 5,387,207 entitled “Thin-Until-Wet Absorbent Foam Materials For Aqueous Body Fluids And Process For Making Same” issued to Dyer et al. on Feb. 7, 1995; and U.S. Pat. No. 5,625,222 entitled “Absorbent Foam Materials For Aqueous Fluids Made From High Internal Phase Emulsions Having Very High Water-To-Oil Ratios” issued to DesMarais et al. on Jul. 22, 1997.

The diaper 20 may also include a fastening system 40. The fastening system 40 preferably maintains the first waist region 36 and the second waist region 38 in a configuration so as to provide lateral tensions about the circumference of the diaper 20 to hold the diaper 20 on the wearer. The fastening system 40 preferably comprises a fastener such as tape tabs, hook and loop fastening components, interlocking fasteners such as tabs & slots, buckles, buttons, snaps, and/or hermaphroditic fastening components, although any other known fastening means are generally acceptable. Some exemplary surface fastening systems are disclosed in U.S. Pat. No. 3,848,594 entitled “Tape Fastening System for Disposable Diaper” issued to Buell on Nov. 19, 1974; U.S. Pat. No. B1 4,662,875 entitled “Absorbent Article” issued to Hirotsu et al. on May 5, 1987; U.S. Pat. No. 4,846,815 entitled “Disposable Diaper Having An Improved Fastening Device” issued to Scripps on Jul. 11, 1989; U.S. Pat. No. 4,894,060 entitled “Disposable Diaper With Improved Hook Fastener Portion” issued to Nestegard on Jan. 16, 1990; U.S. Pat. No. 4,946,527 entitled “Pressure-Sensitive Adhesive Fastener And Method of Making Same” issued to Battrell on Aug. 7, 1990; the herein before referenced U.S. Pat. No. 5,151,092 issued to Buell on Sep. 9, 1992; and U.S. Pat. No. 5,221,274 issued to Buell on Jun. 22, 1993. An exemplary interlocking fastening system is disclosed in co-pending U.S. Pat. No. 6,432,098 entitled “Absorbent Article Fastening Device” in the names of Kline et al. issued on Aug. 13, 2002. The fastening system 40 may also provide a means for holding the article in a disposal configuration as disclosed in U.S. Pat. No. 4,963,140 issued to Robertson et al. on Oct. 16, 1990. The fastening system may also include primary and secondary fastening systems, as disclosed in U.S. Pat. No. 4,699,622 entitled “Disposable Diaper Having An Improved Side Closure” issued to Toussant et al. on Oct. 13, 1987.

### Buckling Force Test Method

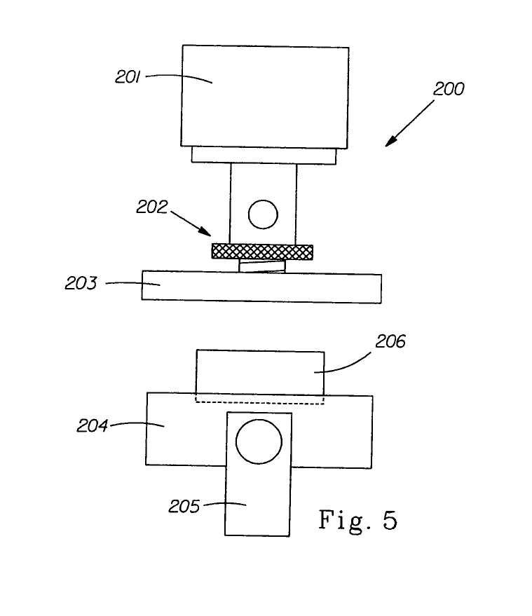

The buckling force was performed on a constant rate of extension tensile tester with computer interface configured for a compression test. The instrument used was a MTS Alliance using Testworks 4.0 Software, as available from MTS Systems Corp., Eden Prairie, Minn. The general setup is shown as 200 in FIG. 5.

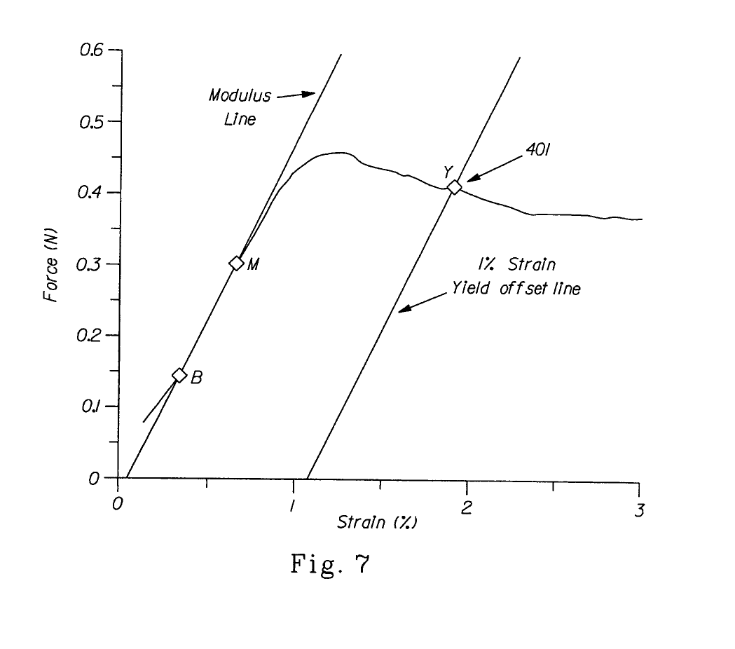

A 10 Newton (N) load cell 201 was used for all measurements. The bottom stationary, pneumatic jaw 205 was fitted with two 3 inch×1 inch diamond faced grips 204, and a 3 inch diameter compression platen 203 with locking collar 202 used as the upper movable fixture. The gage length was set to 30 cm. The platen 203 was lowered at 10 mm/min to a preload 0.05 N, the gage length was then corrected (i.e. reset to the gage where the platen was just touching the top of sample 206) and data was collected as the platen was lowered at 5 mm/min to 50% strain. The buckling force was calculated from the Force vs. Strain curve as the force at the 1% strain yield offset, shown graphically as 401 in FIG. 7.

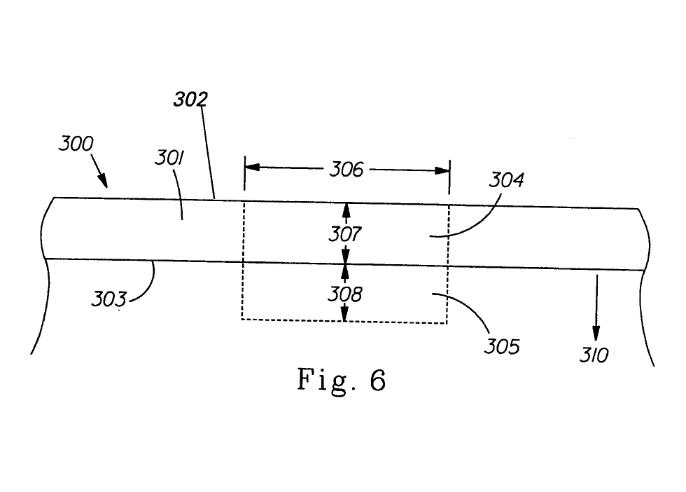

The test sample of the externally visible, serviceable indicia and the test sample immediately adjacent inboard to the indicia sample are shown in FIGS. 6 as 304 and 305, respectively. All dimensions were measured using a calibrated ruler or caliper that could measure to at least the nearest 0.5 mm. The diaper sample was placed flat, backsheet up, on a bench top. If the serviceable indicium 301 was slightly curved, outboard edge 302 was straightened out by minimally stretching the indicia to provide a straight edge for the compression test. A sample strip was prepared by making two parallel cuts, perpendicular to the long direction of the indicia 50 mm apart (distance 306), extending into the diaper about 2.5 times the height (i.e. 2.5×distance 307) of the serviceable indicia 301. This strip was cut along the inboard edge of the indicia 303, parallel to the outboard edge 302 to provide indicia specimen 304. The height 307 of the indicia specimen 304 was measured and the adjacent specimen 305 was cut such that its height 308 was the same as the height 307 of the indicia specimen 305. Any layered components within the adjacent specimen were not removed and were tested as part of the specimen.

The orientation of the two specimens was maintained as they were in the diaper for testing. The edge farthest from the interior 310 of the diaper (i.e. the distal edge) was the edge tested against the movable platen 203, while the edge closest to the interior 310 of the diaper (i.e. the proximal edge) was placed in the grip faces 204 of the stationary fixture.

A line 3 mm from the proximal edge of the indicia specimen 304 was measured and marked across the entire width 306 of the specimen and parallel to the distal edge. For specimens that were slightly curved, care was taken that the marked line was parallel to the distal edge of the specimen. The proximal edge of the indicia specimen 304 was placed into the grip faces 304, aligning the specimen with the marked line (i.e. 3 mm of the specimen in the grips). If necessary, the specimen was stretched minimally so that the distal edge was parallel to the movable platen 203 across the complete width of the specimen. The grip faces were closed and the distal edge of the indicia specimen 304 was checked to verify that it was parallel to the movable platen 203, and the specimen was standing upright, perpendicular to the horizontal. The buckling force test was then performed on the indicia specimen 304 as described above.

Next, adjacent specimen 305 was tested in the same fashion as the indicia specimen 304. A line 3 mm from the proximal edge of the adjacent specimen 305 was measured and marked across the entire width 306 of the specimen, parallel to its distal edge. The proximal edge was placed into the grip faces 204, the specimen was aligned with the marked line (i.e. 3 mm of the specimen in the grips), and then the grip faces were closed. The distal edge of the adjacent specimen 305 was checked to assure that it was parallel to the movable platen 203 and was standing perpendicular to the horizontal. The buckling force test was then performed on the adjacent specimen 305 as described above.

### EXAMPLE

A prototype diaper product in accordance with the present invention was tested per the aforementioned buckling force test method. The prototype diaper included stretchable side panels separately attached in the rear waist region forming, in combination with the end edge and longitudinal side edges, a waist opening and two leg openings. The prototype diaper product included serviceable indicia along an end edge in the back of the product including the side panel, an end edge in the front of the product and along opposing longitudinal side edges of the product including the side panel. The serviceable indicia along each of the edges were about 0.60 inches wide. For the end edge in the back of the product, the buckling force was measured at two locations, a center portion near the longitudinal centerline of the product and an outboard portion along the side panel (ear) of the product. For the end edge in the front of the product, the buckling force was measured in the center portion along the longitudinal axis. For serviceable indicia along the longitudinal side edges, the buckling force was measured at two locations along one of the longitudinal side edges. One buckling force measurement was taken in the crotch region of the longitudinal side edge and another was taken near the side panel (ear). For each case, buckling force was measured for the structure including the serviceable indicia and the structure inboard of and adjacent to the serviceable indicia.

End Edge in Back (Center Portion)

The cross section of structure including the serviceable indicia in the center portion of the end edge in the back of the product included BBA Nonwovens HEC 27 gsm backsheet nonwoven, Clopay BR121 28 gsm backsheet film, BBA Nonwovens 18.6 gsm spunbond nonwoven topsheet material and two serviceable indicia layers (one on the interior surface and one on the exterior surface). Each serviceable indicia layer included a corrugated (gathered) laminate comprising 25 gsm extruded strand elastics as measured in the relaxed state (Kuraray elastomer PG018-Arman Ashraf with Bostik Findley H2401 9 gsm adhesive) sandwiched between one external layer of 17 gsm PGI nonwoven (GCAS#95244615, Lot#M01609) and one internal layer of 12 gsm BBA spunbond nonwoven. The extruded strand elastics were stretched 2.5 times their relaxed length during lamination and then relaxed forming the corrugations. The cross section of the structure immediately adjacent the serviceable indicia included the same materials except for the materials forming the serviceable indicia.

End Edge in Back Along Side Panel (Ear)

The cross section of structure including the serviceable indicia in the outboard portion of the end edge along the side panel (ear) in the back of the product included side panel material comprising two layers of BBA Nonwovens HEC 31 gsm nonwoven sandwiching 1 layer of Nordenia 65 micron thick elastomeric flat film and two serviceable indicia layers (one on the interior surface and one on the exterior surface). Each serviceable indicia layer included a corrugated (gathered) laminate comprising 25 gsm extruded strand elastics as measured in the relaxed state (Kuraray elastomer PG018-Arman Ashraf with Bostik Findley H2401 9 gsm adhesive) sandwiched between one external layer of 17 gsm PGI nonwoven (GCAS#95244615, Lot#M01609) and one internal layer of 12 gsm BBA spunbond nonwoven. The extruded strand elastics were stretched 2.5 times their relaxed length during lamination and then relaxed forming the corrugations. The cross section of the structure in board of and adjacent to the serviceable indicia included the same materials except for the materials forming the serviceable indicia.

End Edge in Front (Center Portion)

The cross section of structure including the serviceable indicia in the center portion of the end edge in the front of the product included BBA Nonwovens HEC 27 gsm backsheet nonwoven, Clopay BR121 28 gsm backsheet film, BBA Nonwovens 18.6 gsm spunbond nonwoven topsheet material and two serviceable indicia layers (one on the interior surface and one on the exterior surface). Each serviceable indicia layer included a corrugated (gathered) laminate comprising 25 gsm extruded strand elastics as measured in the relaxed state (Kuraray elastomer PG018-Arman Ashraf with Bostik Findley H2401 9 gsm adhesive) sandwiched between one external layer of 17 gsm PGI nonwoven (GCAS#95244615, Lot#M01609) and one internal layer of 12 gsm BBA spunbond nonwoven. The extruded strand elastics were stretched 2.5 times their relaxed length during lamination and then relaxed forming the corrugations. The cross section of the structure in board of and adjacent to the serviceable indicia included the same materials except for the materials forming the serviceable indicia. This portion also included a layer of Nordenia 85 gsm brushed knit landing zone material.

Opposing Longitudinal Side Edges (Crotch Area and Near Side Panel)

The serviceable indicia along the opposing longitudinal side edges in the crotch area and near the side panel (ear) comprised a c-folded layer forming a finished outer leg cuff. The cross section of structure including the serviceable indicia along the opposing longitudinal side edges of the product included BBA Nonwovens HEC 27 gsm backsheet nonwoven, Clopay BR121 28 gsm backsheet film, Corovin 17 gsm spunbond meltblown spunbond nonwoven cuff material and two serviceable indicia layers (one on the interior surface and one on the exterior surface). Each serviceable indicia layer included a corrugated (gathered) laminate comprising 25 gsm extruded strand elastics as measured in the relaxed state (Kuraray elastomer PG018-Arman Ashraf with Bostik Findley H2401 9 gsm adhesive) sandwiched between one external layer of 17 gsm PGI nonwoven (GCAS#95244615, Lot#M01609) and one internal layer of 12 gsm BBA spunbond nonwoven. The extruded strand elastics were stretched 2.5 times their relaxed length during lamination and then relaxed forming the corrugations. The cross section of the structure in board of and adjacent to the serviceable indicia included the same materials except for the materials forming the serviceable indicia.

The results of the buckling test performed on the prototype are summarized below.
Prototype Diaper Product

Individual Measurements

| | 1st Replicate | | 2nd Replicate | |
|---|---|---|---|---|
| | Force | | Force | |
| | @ Yield | Indicia/ | @ Yield | Indicia/ |
| | (N) | Adjacent | (N) | Adjacent |
| Front Waist (center)—Indicia | 1.9 | 1.5 | | |
| Front Waist (center)—Adjacent | 1.3 | | | |
| Back Waist (center)—Indicia | 4.4 | 14.7 | | |
| Back Waist (center)—Adjacent | 0.3 | | | |
| Back Waist (ear)—Indicia | 3.6 | 12.0 | | |
| Back Waist (ear)—Adjacent | 0.3 | | | |
| Cuff (near ear)—Indicia | 3.0 | 30.0 | 2.9 | 14.5 |
| Cuff (near ear)—Adjacent | 0.1 | | 0.2 | |
| Cuff (crotch)—Indicia | 4.5 | 11.3 | 4.1 | 13.7 |
| Cuff (crotch)—Adjacent | 0.4 | | 0.3 | |

Average Measurements

| | Force @ | Force @ | Indicia/ |
|---|---|---|---|
| | Yield (N) | Yield (N) | Adjacent |
| | Indicia | Adjacent | Ratio |
| Front Waist | 1.9 | 1.3 | 1.5 |
| Back Waist | 4.0 | 0.3 | 13.3 |
| Cuff | 3.6 | 0.3 | 14.5 |

For comparison purposes, the buckling force test method was also performed on the end edges and opposing longitudinal side edges of current market products. Two diapers were tested per each current market product. The products and the corresponding test results are summarized in the tables below.

### Pampers Easy Ups, Size 3T/4T—Lot 431901143

Individual Measurements

| | 1st Replicate | | 2nd Replicate | |
|---|---|---|---|---|
| | Force @ | Indicia/ | Force @ | Indicia/ |
| | Yield (N) | Adjacent | Yield (N) | Adjacent |
| Front Waist—Indicia | 1.50 | 4.3 | 0.87 | 3.3 |
| Front Waist—Adjacent | 0.35 | | 0.26 | |
| Back Waist—Indicia | 1.59 | 2.7 | 0.62 | 1.5 |
| Back Waist—Adjacent | 0.60 | | 0.41 | |
| Cuff—Indicia | 0.40 | 4.0 | 0.50 | 6.3 |
| Cuff—Adjacent | 0.10 | | 0.08 | |
| Cuff—Indicia | 0.43 | 2.0 | 0.40 | 2.5 |
| Cuff—Adjacent | 0.22 | | 0.16 | |

Average Measurements

| | Force @ | Force @ | Indicia/ |
|---|---|---|---|
| | Yield (N) | Yield (N) | Adjacent |
| | Indicia | Adjacent | Ratio |
| Front Waist | 1.19 | 0.31 | 3.9 |
| Back Waist | 1.11 | 0.51 | 2.2 |
| Cuff | 0.43 | 0.14 | 3.1 |

### Huggies Pull Ups, Size 2T/3T—Lot PA410122X

Individual Measurements

| | 1st Replicate | | 2nd Replicate | |
|---|---|---|---|---|
| | Force @ | Indicia/ | Force @ | Indicia/ |
| | Yield (N) | Adjacent | Yield (N) | Adjacent |
| Front Waist—Indicia | 1.30 | 162.5 | 0.67 | 11.2 |
| Front Waist—Adjacent | 0.01 | | 0.06 | |
| Back Waist—Indicia | 1.64 | 20.5 | 0.03 | 0.8 |
| Back Waist—Adjacent | 0.08 | | 0.04 | |
| Cuff—Indicia | 0.05 | 0.8 | 0.04 | 0.4 |
| Cuff—Adjacent | 0.06 | | 0.11 | |
| Cuff—Indicia | 0.03 | 0.4 | 0.07 | 1.4 |
| Cuff—Adjacent | 0.08 | | 0.05 | |

Average Measurements

| | Force @ | Force @ | Indicia/ |
|---|---|---|---|
| | Yield (N) | Yield (N) | Adjacent |
| | Indicia | Adjacent | Ratio |
| Front Waist | 0.99 | 0.03 | 29.0 |
| Back Waist | 0.84 | 0.06 | 13.9 |
| Cuff | 0.05 | 0.08 | 0.6 |

### Huggies Supremes, Size 5—Lot PA413507F (Soft Knit Elastics)

Individual Measurements

| | 1st Replicate | | 2nd Replicate | |
|---|---|---|---|---|
| | Force @ | Indicia/ | Force @ | Indicia/ |
| | Yield (N) | Adjacent | Yield (N) | Adjacent |
| Front Waist—Indicia | 0.14 | 0.7 | 0.54 | 0.9 |
| Front Waist—Adjacent | 0.20 | | 0.58 | |
| Back Waist—Indicia | 0.49 | 16.3 | 0.16 | 5.3 |
| Back Waist—Adjacent | 0.03 | | 0.03 | |
| Cuff—Indicia | 0.66 | 5.5 | 0.49 | 8.2 |
| Cuff—Adjacent | 0.12 | | 0.06 | |
| Cuff—Indicia | 0.19 | 19.0 | 0.21 | 7.0 |
| Cuff—Adjacent | 0.01 | | 0.03 | |

Average Measurements

| | Force @ | Force @ | Indicia/ |
|---|---|---|---|
| | Yield (N) | Yield (N) | Adjacent |
| | Indicia | Adjacent | Ratio |
| Front Waist | 0.34 | 0.39 | 0.9 |
| Back Waist | 0.33 | 0.03 | 10.8 |
| Cuff | 0.39 | 0.06 | 7.0 |

### Huggies Supremes, Size 4—Lot 615100126

Individual Measurements

| | 1st Replicate | | 2nd Replicate | |
|---|---|---|---|---|
| | Force @ | Indicia/ | Force @ | Indicia/ |
| | Yield (N) | Adjacent | Yield (N) | Adjacent |
| Front Waist—Indicia | 0.53 | 1.6 | 0.47 | 1.9 |
| Front Waist—Adjacent | 0.34 | | 0.25 | |
| Back Waist—Indicia | 0.56 | 11.2 | 0.81 | 6.8 |
| Back Waist—Adjacent | 0.05 | | 0.12 | |
| Cuff—Indicia | 0.22 | 7.3 | 0.08 | 1.6 |
| Cuff—Adjacent | 0.03 | | 0.05 | |
| Cuff—Indicia | 0.33 | 6.6 | 0.26 | 6.5 |
| Cuff—Adjacent | 0.05 | | 0.04 | |

Average Measurements

| | Force @ | Force @ | Indicia/ |
|---|---|---|---|
| | Yield (N) | Yield (N) | Adjacent |
| | Indicia | Adjacent | Ratio |
| Front Waist | 0.50 | 0.30 | 1.7 |
| Back Waist | 0.69 | 0.09 | 8.1 |
| Cuff | 0.22 | 0.04 | 5.2 |

### Pampers Cruisers, Size 4—Lot 5188U01131

Individual Measurements

| | 1st Replicate | | 2nd Replicate | |
|---|---|---|---|---|
| | Force @ | Indicia/ | Force @ | Indicia/ |
| | Yield (N) | Adjacent | Yield (N) | Adjacent |
| Front Waist—Indicia | 0.06 | 0.2 | 0.03 | 0.1 |
| Front Waist—Adjacent | 0.25 | | 0.57 | |
| Back Waist—Indicia | 0.14 | 2.3 | 0.05 | 1.3 |
| Back Waist—Adjacent | 0.06 | | 0.04 | |
| Cuff—Indicia | 0.12 | 0.7 | 0.10 | 0.7 |
| Cuff—Adjacent | 0.17 | | 0.14 | |
| Cuff—Indicia | 0.12 | 1.1 | 0.24 | 4.8 |
| Cuff—Adjacent | 0.11 | | 0.05 | |

Average Measurements

| | Force @ | Force @ | Indicia/ |
|---|---|---|---|
| | Yield (N) | Yield (N) | Adjacent |
| | Indicia | Adjacent | Ratio |
| Front Waist | 0.05 | 0.41 | 0.1 |
| Back Waist | 0.10 | 0.05 | 1.9 |
| Cuff | 0.15 | 0.12 | 1.2 |

All documents cited in the Detailed Description of the Invention are, in relevant part, incorporated herein by reference; the citation of any document is not to be construed as an admission that it is prior art with respect to the present invention. To the extent that any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a lateral axis;
a longitudinal axis;
a topsheet;
a backsheet;
an absorbent core disposed between the topsheet and the backsheet;
a first waist region on a first side of the lateral axis;
a second waist region opposed to the first waist region and on a second side of the lateral axis;
an elastic waist member in the second waist region and joined to a portion of the topsheet, wherein the elastic waist member is laterally wider than the absorbent core;
an elasticized side panel in the second waist region, wherein the side panel comprises a discrete web, and wherein the elasticized side panel extends laterally outward relative to a portion of the elastic waist member;
a member joined to a laterally outboard portion of the side panel, wherein the member comprises a first portion positioned most proximal to the longitudinal axis, a second portion positioned most distal from the longitudinal axis, and a middle portion positioned between the first and second portions of the member, and wherein the member comprises a different material than the side panel; and
a fastener comprising hooks joined to the member in the middle portion on a wearer-facing surface of the member, wherein the first and second portions of the member are free of the fastener, and wherein the first portion of the member has a larger lateral width than the second portion of the member;
wherein the second portion of the member comprises an arcuate portion;
wherein a portion of a first edge of the middle portion of the member positioned most distal from the lateral axis of the absorbent article is angled relative to the lateral axis when viewing the wearer-facing surface of the absorbent article; and
wherein a portion of a second edge of the middle portion of the member positioned most proximate to the lateral axis of the absorbent article is angled relative to the lateral axis when viewing the wearer-facing surface of the absorbent article.

2. The absorbent article of claim 1, wherein the second portion of the member has a smaller longitudinal length, in a direction parallel to the longitudinal axis of the absorbent article, than a longitudinal length of the first portion of the member.

3. The absorbent article of claim 1, wherein the portion of the first edge of the middle portion of the member has a negative slope relative to the lateral axis when viewing the wearer-facing surface of the absorbent article, and wherein the portion of the second edge of the middle portion of the member has a positive slope relative to the lateral axis when viewing the wearer-facing surface of the absorbent article.

4. The absorbent article of claim 1, wherein the fastener fully covers the middle portion of the member, and wherein the backsheet extends to the side panel.

5. An absorbent article comprising:
a lateral axis;
a longitudinal axis;
a topsheet;
a backsheet;
an absorbent core disposed between the topsheet and the backsheet;
a first waist region on a first side of the lateral axis;
a second waist region opposed to the first waist region and on a second side of the lateral axis;
an elastic waist member in the second waist region, wherein the elastic waist member is laterally wider than the absorbent core;
an elasticized side panel in the second waist region, wherein the side panel comprises a separate web, and wherein the elasticized side panel extends laterally outward relative to a portion of the elastic waist member;
a member joined to a laterally outboard portion of the side panel, wherein the member comprises a first portion positioned most proximal to the longitudinal axis, a second portion positioned most distal from the longitudinal axis, and a middle portion positioned between the first and second portions of the member, and wherein the member comprises a different material than the side panel; and
a fastener joined to the member in the middle portion, wherein the first portion and the second portion of the member are free of the fastener, and wherein the first portion of the member has a larger lateral width than the second portion of the member;
wherein the second portion of the member comprises an arcuate outer portion;
wherein a portion of a first edge of the middle portion of the member positioned most distal from the lateral axis of the absorbent article is angled relative to the lateral axis of the absorbent article when viewing the wearer-facing surface of the absorbent article;
wherein a portion of a second edge of the middle portion of the member positioned most proximal to the lateral axis of the absorbent article is angled relative to the lateral axis when viewing the wearer-facing surface of the absorbent article;
wherein a portion of a first edge of the second portion of the member positioned most distal from the lateral axis of the absorbent article is angled relative to the lateral axis of the absorbent article when viewing the wearer-facing surface of the absorbent article; and
wherein a portion of a second edge of the second portion of the member positioned most proximal to the lateral axis of the absorbent article is angled relative to the lateral axis when viewing the wearer-facing surface of the absorbent article.

6. An absorbent article comprising:
a longitudinal axis;
a lateral axis;
a topsheet;
a backsheet;
an absorbent core interposed between the topsheet and the backsheet;
a first waist region on a first side of the lateral axis;
a second waist region opposed to the first waist region and on a second side of the lateral axis;
an elastic waist feature in the second waist region and joined to a portion of a wearer-facing surface of the absorbent article, wherein the elastic waist feature is configured to elastically expand and contract;
a side panel in the second waist region, wherein a laterally inboard portion of the side panel is joined to a portion of the second waist region;
a nonelastic extension member joined to a laterally outboard portion of the side panel, wherein the nonelastic extension member comprises a first portion positioned most proximal to the longitudinal axis of the absorbent article, a second portion positioned most distal from the longitudinal axis of the absorbent article, and a middle portion disposed between the first portion and second portion, and wherein the first portion has a larger area than the second portion; and
a fastener joined to the nonelastic extension member in the middle portion on a wearer-facing surface of the nonelastic extension member, wherein the first portion and the second portion of the nonelastic extension member are free of the fastener;
wherein the second portion of the nonelastic extension member comprises an arcuate portion; and
wherein a portion of a first edge of the middle portion of the nonelastic extension member positioned most distal from the lateral axis has a negative slope relative to the lateral axis when viewing the wearer-facing surface of the absorbent article.

7. The absorbent article of claim 6, wherein a laterally outboard edge of the elastic waist feature touches the laterally inboard portion of the side panel.

8. The absorbent article of claim 6, wherein a laterally outboard edge of the elastic waist feature is positioned proximate to the laterally inboard portion of the side panel.

9. The absorbent article of claim 6, wherein the absorbent article comprises a chassis and a second side panel in the first waist region.

10. The absorbent article of claim 9, wherein a portion of the second side panel is integrally formed with the chassis.

11. The absorbent article of claim 10, comprising leg cuffs, wherein a portion of the elastic waist feature overlaps a portion of the topsheet and a portion of the backsheet.

12. The absorbent article of claim 6, wherein the fastener covers the entire middle portion of the nonelastic extension member, and wherein the fastener comprises hooks.

13. The absorbent article of claim 6, wherein a portion of a second edge of the middle portion of the nonelastic extension member positioned most proximal to the lateral axis of the absorbent article has a slope relative to the lateral axis of the absorbent article when viewing the wearer-facing surface of the absorbent article.

14. The absorbent article of claim 13, wherein the slope is negative.

15. The absorbent article of claim 13, wherein the slope is positive.

16. The absorbent article of claim 6, wherein the side panel comprises a different material as the nonelastic extension member.

17. The absorbent article of claim 6, wherein the side panel comprises an elasticized portion.

18. An absorbent article comprising:
a lateral axis;
a longitudinal axis;
a topsheet;
a backsheet;
an absorbent core interposed between the topsheet and the backsheet;
a first waist region;
a second waist region opposed to the first waist region;
an elastic waist member in the second waist region and joined to a portion of a wearer-facing surface of the absorbent article;
a side panel in the second waist region, wherein the side panel comprises a discrete web, and wherein a laterally inboard portion of the discrete web is joined to a portion of the wearer-facing surface;
a member joined to a laterally outboard portion of the side panel, wherein the member comprises a first portion positioned most proximal to the longitudinal axis of the absorbent article, a second portion positioned most distal from the longitudinal axis of the absorbent article, and a middle portion disposed between the first portion and the second portion of the member; and
a fastener comprising hooks joined to the member in the middle portion on a wearer-facing surface of the member, wherein the first portion of the member and the second portion of the member are free of the fastener, and wherein the first portion has a larger area than the second portion;
wherein the second portion of the member comprises an arcuate outer portion;
wherein a portion of a first edge of the middle portion of the member positioned most distal from the lateral axis of the absorbent article has a slope relative to the lateral axis when viewing the wearer-facing surface of the absorbent article; and
wherein a portion of a second edge of the middle portion of the member positioned most proximal to the lateral axis of the absorbent article has a slope relative to the lateral axis when viewing the wearer-facing surface of the absorbent article.

19. The absorbent article of claim 18, wherein the member comprises a nonelastic member.

20. The absorbent article of claim 18, wherein the member comprises an extension member.

21. The absorbent article of claim 18, wherein the portion of the first edge of the middle portion of the member has a negative slope relative to the lateral axis when viewing the wearer-facing surface of the absorbent article.

22. The absorbent article of claim 18, wherein the portion of the second edge of the middle portion of the member has a negative slope when viewing the wearer-facing surface of the absorbent article.

23. The absorbent article of claim 18, wherein the portion of the second edge of the middle portion of the member has a positive slope when viewing the wearer-facing surface of the absorbent article.

24. The absorbent article of claim 18, wherein the second edge of the middle portion of the member has a portion having a positive slope and a portion having a negative slope when viewing the wearer-facing surface of the absorbent article.

25. The absorbent article of claim 18, wherein a laterally outboard edge of the elastic waist member touches the laterally inboard portion of the separate web.

26. The absorbent article of claim 18, wherein a laterally outboard edge of the elastic waist feature is positioned proximate to the laterally inboard portion of the separate web.

27. The absorbent article of claim 18, wherein the absorbent article comprises a chassis and a second side panel in the first waist region.

28. The absorbent article of claim 27, wherein a portion of the second side panel is integrally formed with the chassis.

29. The absorbent article of claim 18, comprising leg cuffs, wherein the middle portion of the member is fully overlapped by the fastener.

30. The absorbent article of claim 18, wherein the separate web comprises a different material as the member, and wherein the backsheet extends to the side panel, and wherein a portion of the elastic waist member overlaps a portion of the side panel.
